(19) United States
(12) Patent Application Publication
(10) Pub. No.: US 2018/0325869 A1
(43) Pub. Date: Nov. 15, 2018

(54) TARGETING OF THE FORMYL-PEPTIDE RECEPTOR 2/LIPOXIN A4 RECEPTOR (FPR2/ALX) FOR TREATMENT OF HEART DISEASE

(71) Applicant: BRISTOL-MYERS SQUIBB COMPANY, Princeton, NJ (US)

(72) Inventors: Jacek Ostrowski, Jamison, PA (US); Ricardo Garcia, Ewing, NJ (US); Nicholas R. Wurtz, Pennington, NJ (US)

(21) Appl. No.: 15/776,927

(22) PCT Filed: Nov. 21, 2016
(86) PCT No.: PCT/US2016/063036

§ 371 Date: May 17, 2018

Related U.S. Application Data
(60) Provisional application No. 62/259,498, filed on Nov. 24, 2015.

(51) Int. Cl. A61K 31/4152; A61P 9/10; A61P 9/04

(52) U.S. Cl. CPC A61K 31/4152; A61P 9/04; A61P 9/10

(57) ABSTRACT

The disclosure generally relates to a therapeutic approach based on the stimulation of resolution of inflammation by the Formyl-Peptide Receptor 2/Lipoxin A₄ receptor (FPR2/ALX) for the treatment of heart disease.

### CROSS REFERENCE TO RELATED APPLICATION

This non-provisional application claims the benefit of U.S. Provisional Application Ser. No. 62/259,498 filed Nov. 24, 2015 which is herein incorporated by reference in its entirety.

### BACKGROUND OF THE INVENTION

This disclosure describes a therapeutic approach which based on the stimulation of resolution of inflammation by the Formyl-Peptide Receptor 2/Lipoxin A₄ receptor (FPR2/ALX) for the treatment of heart disease.

Heart disease is an increasingly prevalent condition that exerts a significant clinical and economic burden. The increase in prevalence is driven by patients surviving myocardial infarctions leading to cumulative myocardial damage that progressively leads to adverse cardiac remodeling and left ventricular dysfunction (Viau D M et al., Heart, 2015, 101, 1862-7., Paulus W J., Tschope C., J. Am. Coll. Cardiol., 2013, 62, 263-71). Despite the growing prevalence and social burden of this disease, there have been very few, if any, recent advances in treatment. Standard of care for acute coronary syndrome (ACS) patients after PCI includes aspirin, statins, beta-blockers, and ACE inhibitor/ARB therapies (Zouein F A et al., J. Cardiovasc. Pharmacol., 2013, 62, 13-21).

Formyl peptide receptors 2/lipoxin A₄ (FPR2/ALX) belongs to small group of seven-transmembrane domain, G protein-coupled receptors that are expressed mainly by mammalian phagocytic leukocytes and are known to be important in host defense and inflammation. The FPR2/ALX share significant sequence homology with FPR1 and FPR3. Collectively, these receptors bind a number of structurally diverse group of agonists, including N-formyl and nonformyl peptides which act as chemo attractants and activate phagocytes. The endogenous peptide annexin 1 and its N-terminal fragments also bind human FPR1 and FPR2/ALX. Importantly, eicosanoid lipoxin A₄, which belongs to newly discovered class of small pro-resolution mediators (SPMs), has been recently identified as specific agonist for the FPR2 (Ye RD., et al., Pharmacol. Rev., 2009, 61, 119-61).

Endogenous FPR2/ALX pro-resolution ligands, such as lipoxin A₄, resolving D1 and Annexin A1 bind to the receptor triggering a wide array of cytoplasmatic cascades such as the Gi coupling, Ca²⁺ mobilization and β-arresting recruitment. Activation of FPR2/ALX by lipoxin A₄ modifies the effects of peptidic agonists, such as serum amyloid A (SAA), and has alternative effects on phosphorylation pathways depending on the cell type. Lipoxins regulate components of both innate and adaptive immune systems including neutrophils, macrophages, T-, and B-cells. In neutrophils, lipoxins modulate movement, cytotoxicity and life span. In macrophages, lipoxins prevent apoptosis and enhance efferocytosis. In most inflammatory cells, lipoxins also down-regulate expression of several pro-inflammatory cytokines, such as IL-6, IL-1β and IL-8 as well as up-regulate expression of anti-inflammatory cytokine IL-10 (Chandrasekharan J A, Sharma-alalia N,. J. Inflamm. Res., 2015, 8, 181-92).

The primary effects of lipoxin on neutrophils and macrophages are termination of inflammation and initiation of resolution of inflammation. The latter is primarily responsible for enhancing anti-fibrotic wound healing and returning of the injured tissue to homeostasis (Romano M., et al., Eur. J. Pharmacol., 2015, 5, 49-63). Activation of the FPR2/ALX by endogenous small pro-resolution mediators (SPMs) such as Lipoxin A₄ (LXA4) and synthetic compounds results in stimulation of the non-phlogistic recruitment of monocytes and activation of macrophages in a manner that enhances the efferocytosis of apoptotic cells and promotes the clearance of necrotic cell debris. Stimulation of FPR2/ALX activity also results in suppression of neutrophil recruitment.

In the cardiovascular system both the FPR2/ALX receptor and its pro-resolution agonists were found to be responsible for atherogenic-plaque stabilization and healing (Petri M H., et al., Cardiovasc. Res., 2015, 105, 65-74; and Fredman G., et al., Sci. Trans. Med., 2015, 7(275); 275ra20). Lipoxins and its receptor also have been shown to be beneficial in preclinical models of chronic inflammatory human diseases, including: infectious diseases, psoriasis, dermatitis, ocular inflammation, sepsis, pain, metabolic/diabetes diseases, cancer, COPD, asthma and allergic diseases, cystic fibrosis, acute lung injury and fibrosis, rheumatoid arthritis and other joint diseases, Alzheimer's disease, kidney fibrosis, and organ transplantation (Romano M., et al., Eur. J. Pharmacol., 2015, 5, 49-63, Perrett, M., et al., Trens in Pharm. Sci., 2015, 36, 737-755.)

Chronic inflammation is part of the pathway of pathogenesis of many human diseases and stimulation of resolution pathways with FPR2/ALX agonists may have both protective and reparative effects. Ischaemia-reperfusion (I/R) injury is a common feature of several diseases associated with high morbidity and mortality, such as myocardial infarction and stroke. The non-productive wound healing associated with cardiomyocyte death and pathological remodeling resulting from ischemia-reperfusion injury leads to the scar formation, fibrosis, and progressive loss of heart function. Various aspects of the present invention provide for use of FPR2/ALX agonists in the treatment of heart disease including non-productive wound healing associated with cadiomyocytes death and pathological remodeling which can lead to scar formation, fibrosis, and progressive loss of heart function.

### DESCRIPTION OF THE INVENTION

Various aspects of the present invention describe therapeutic approaches to heart disease which are based on the stimulation of resolution of inflammation by the Formyl-Peptide Receptor 2/Lipoxin A₄ receptor (FPR2/ALX).

Compound 1 is 1-(4-chlorophenyl)-3-(5-isopropyl-1-methyl-3-oxo-2-phenyl-2,3-dihydro-1H-pyrazol-4-yl)urea (Burli, R. W. et al. Biorg. Med. Chem. Lett. 16, 3713-3718 (2006)) and has the following structure:

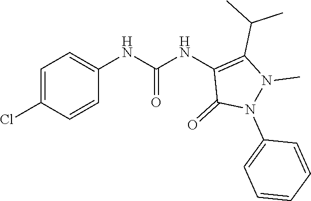 [SMILES: CC(C)c1c(NC(=O)Nc2ccc(Cl)cc2)c(=O)n(-c2ccccc2)n1C]

The invention includes all pharmaceutically acceptable salt forms of the compounds. Pharmaceutically acceptable salts are those in which the counter ions do not contribute significantly to the physiological activity or toxicity of the compounds and as such function as pharmacological equivalents. These salts can be made according to common organic techniques employing commercially available reagents. Some anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate. Some cationic salt forms include ammonium, aluminum, benzathine, bismuth, calcium, choline, diethylamine, diethanolamine, lithium, magnesium, meglumine, 4-phenylcyclohexylamine, piperazine, potassium, sodium, tromethamine, and zinc.

### Pharmaceutical Composition and Methods of Use

The compounds of this invention modulate FPR2/ALX. Accordingly, one aspect of the invention is a method for treating heart disease comprising administering a therapeutically effective amount of an FPR2/ALX agonist to a patient in need thereof

Another aspect of the invention is the method wherein the heart disease is selected from the group consisting of angina pectoris, unstable angina, myocardial infarction, heart failure, acute coronary disease, acute heart failure, chronic heart failure, and cardiac iatrogenic damage.

Another aspect of the invention is the method wherein the heart disease is post myocardial infarction.

Another aspect of the invention is the method wherein the heart disease is associated with chronic heart failure.

Another aspect of the invention is the method wherein the treatment is to improve myocardial wound healing.

Another aspect of the invention is the method wherein the treatment is to improve diminish myocardial fibrosis.

Another aspect of the invention is the method wherein the agonist is 1-(4-chlorophenyl)-3-(5-isopropyl-1-methyl-3-oxo-2-phenyl-2,3-dihydro-1H-pyrazol-4-yl)urea or a pharmaceutically acceptable salt thereof.

“Therapeutically effective” means the amount of agent required to provide a meaningful patient benefit as understood by practitioners in the field of cardiovascular diseases and conditions.

“Patient” means an mammalian species, including humans, with a cardiovascular condition that is suitable for treatment as determined by practitioners in the field of cardiovascular diseases and conditions.

As used herein, “treating” or “treatment” cover a treatment of a disease-state in a mammal, particularly in a human, and include: (a) inhibiting a disease-state, i.e., arresting it development; and/or (b) relieving a disease-state, i.e., causing regression of a disease state; and/or (c) prophylaxis of a disease state. As used herein, “prophylaxis” is the protective treatment of a disease state to reduce and/or minimize the risk and/or reduction in the risk of recurrence of a disease state by administering to a patient a therapeutically effective amount of at least one of the compounds of the present invention or a or a stereoisomer, a tautomer, a pharmaceutically acceptable salt, or a solvate thereof Patients may be selected for prophylaxis therapy based on factors that are known to increase risk of suffering a clinical disease state compared to the general population. For prophylaxis treatment, conditions of the clinical disease state may or may not be presented yet. “Prophylaxis” treatment can be divided into (a) primary prophylaxis and (b) secondary prophylaxis. Primary prophylaxis is defined as treatment to reduce or minimize the risk of a disease state in a patient that has not yet presented with a clinical disease state, whereas secondary prophylaxis is defined as minimizing or reducing the risk of a recurrence or second occurrence of the same or similar clinical disease state.

As used herein, “prevention” cover the preventive treatment of a subclinical disease-state in a mammal, particularly in a human, aimed at reducing the probability of the occurrence of a clinical disease-state. Patients are selected for preventative therapy based on factors that are known to increase risk of suffering a clinical disease state compared to the general population.

In another embodiment, the present invention provides a combined preparation of a compound of the present invention and additional therapeutic agent(s) for simultaneous, separate or sequential use in therapy.

The compounds of the invention may be used with one or more, preferable one to three, of the following heart failure agents selected from loop diuretics, Angiotensin converting enzyme (ACE) inhibitors, Angiotensin II receptor blockers (ARBs), angiotensin receptor-neprilysin inhibitors (ARNI), beta blockers, mineralocorticoid receptor antagonists, nitroxyl donors, RXFP1 agonists, APJ agonists and cardiotonic agents. These agents include, but are not limited to furosemide, bumetanide, torsemide, sacubitrial-valsartan, thiazide diruetics, captopril, enalapril, lisinopril, carvedilol, rnetopolol, bisoproiol, sereiaxin., spironolactone, eplerenone, ivabradine, candesartan, eprosartan, irbestarain, losartan, olmesartan, telmisartan, and valsartan.

Heart disease is a class of diseases which encompasses angina pectoris, unstable angina, myocardial infarction, heart failure, acute coronary disease, acute heart failure, chronic heart failure, and cardiac iatrogenic damage, as well other associated diseases as understood by practitioners in the field of cardiovascular diseases and conditions.

The compounds of this invention are generally given as pharmaceutical compositions comprised of a therapeutically effective amount of an FPR2/ALX compound and a pharmaceutically acceptable carrier and may contain conventional excipients. A therapeutically effective amount is that which is needed to provide a meaningful patient benefit. Pharmaceutically acceptable carriers are those conventionally known carriers having acceptable safety profiles. Compositions encompass all common solid and liquid forms including capsules, tablets, losenges, and powders as well as liquid suspensions, syrups, elixers, and solutions. Compositions are made using common formulation techniques, and conventional excipients (such as binding and wetting agents) and vehicles (such as water and alcohols) are generally used for compositions. See, for example, Remington's Pharmaceutical Sciences, 17th edition, Mack Publishing Company, Easton, Pa. (1985).

Solid compositions are normally formulated in dosage units and compositions providing form about 1 to 1000 mg of the active ingredient per dose are preferred. Some examples of dosages are 1 mg, 10 mg, 100 mg, 250 mg, 500 mg, and 1000 mg.

Liquid compositions are usually in dosage unit ranges. Generally, the liquid composition will be in a unit dosage range of 1-100 mg/mL. Some examples of dosages are 1 mg/mL, 10 mg/mL, 25 mg/mL, 50 mg/mL, and 100 mg/mL.

The invention encompasses all conventional modes of administration; oral and parenteral methods are preferred. Generally, the dosing regimen will be similar to other cardiovascular agents used clinically. Typically, the daily dose will be 0.1-100 mg/kg body weight daily. Generally, more compound is required orally and less parenterally. The specific dosing regimen, however, will be determined by a physician using sound medical judgment.

### Biological Methods and Results

FPR2 and FPR1 cAMP assays. A mixture of forskolin (5 μM final for FPR2/ALX or 10 μM final for FPR1) and IBMX (200 μM final) were added to 384-well Proxiplates (Perkin-Elmer) pre-dotted with test compounds in DMSO (1% final) at final concentrations in the range of 1.7 nM to 100 μM. Chinese Hamster Ovary cells (CHO) overexpressing human FPR1 or human FPR2/ALX receptors were cultured in F-12 (Ham's) medium supplemented with 10% qualified FBS, 250 μg/ml zeocin and 300 μg/ml hygromycin (Life Technologies). Reactions were initiated by adding 2,000 human FPR2 cells per well or 4,000 human FPR1 cells per well in Dulbecco's PBS (with calcium and magnesium) (Life Technologies) supplemented with 0.1% BSA (Perkin-Elmer). The reaction mixtures were incubated for 30 min at room temperature. The level of intracellular cAMP was determined using the HTRF HiRange cAMP assay reagent kit (Cisbio) according to manufacturer's instruction. Solutions of cryptate conjugated anti-cAMP and d2 flurorophore-labelled cAMP were made in a supplied lysis buffer separately. Upon completion of the reaction, the cells were lysed with equal volume of the d2-cAMP solution and anti-cAMP solution. After a 1-h room temperature incubation, time-resolved fluorescence intensity was measured using the Envision (Perkin-Elmer) at 400 nm excitation and dual emission at 590 nm and 665 nm. A calibration curve was constructed with an external cAMP standard at concentrations ranging from 1 μM to 0.1 pM by plotting the fluorescent intensity ratio from 665 nm emission to the intensity from the 590 nm emission against cAMP concentrations. The potency and activity of a compound to inhibit cAMP production was then determined by fitting to a 4-parametric logistic equation from a plot of cAMP level versus compound concentrations.

Flipr assay using dHL60 non-adherent cell line. HL60 cells were diluted to 1.5×10⁵ cells/ml and were grown in culture medium containing 1.3% DMSO at 37C for 5 days. On day 6 cells were counted to make sure that cells viability was approx. 95%. The 1.2×10⁷ cells were spin down and washed cells once with assay buffer. The supernatant was removed and cells were re-suspended in 12 ml buffer with fluo-4 AM loading dye and label cells at 37C for 30 min. Loading buffer: HBSS (invitrogen, cat 14075), 20 mM HEPES, 0.1% FAF-BSA, 15 ul of 0.025% pluronic F127 (Invitrogen, P3000 MP), 2.5 mM probenecid, 1.9 uM Fluo-4 AM (Invitrogen, F14201). After incubation cells were washed once with reaction buffer to remove the dye and were re-suspended at 1×10⁶ cells/ml. Following wash, cells were plated in 100 ul/well in Poly-D-Lysine pre-coated 96 well assay plates. Assay plates were centrifuged at 1000 rpm for 10 min and then placed in the FLIPR to perform calcium flux assay.

β-arrestin recruitment assay. DiscoveRx standard protocol was used.

HL-60 cell culture and differentiation. The HL-60 cell line (ATCC, CCL-240, lot 60398411) was maintained in IMDM (Life Tech, cat 12440-053) medium supplemented with 20% fetal bovine serum, 50 U/ml penicillin, and 50 μg/ml streptomycin at 37° with 5% CO₂. Cells were differentiated into the granulocyte lineage with DMSO; 2.5×10⁵ cells/ml were incubated with 1.25% DMSO for 5 days.

Neutrophil and HL-60 cell migration assay agonist mode. After 5 day differentiation, cells were resuspended in phenol free RPMI (Invitrogen, cat 11835) with 0.2% fatty acid free BSA at a concentration of 3×10⁷ cells/ml. The dHL-60 cells (10⁵ in 100 μl) were added to the upper chamber of each HTS transwell-96well plate (Corning#3387). Migration was induced by placing chemoattractant in the bottom chamber and the dHL60 cells in the top chamber of the transwell plate. Cells were allowed to migrate for 120 min across the 5 micron filters at 37° with 5% CO₂. Following migration, neutrophils or dHL-60 cells remaining in the transwell lower chamber (migrated fraction) were quantitated using the cell-titer-glo luminescence cell viability assay (Promega, G7571).

Neutrophil and HL-60 cell migration assay antagonist mode. After 5 day differentiation, the cells were resuspended in phenol free RPMI (Invitrogen, cat 11835) with 0.2% fatty acid free BSA at a concentration of 3×10⁷ cells/ml. The dHL-60 cells (10⁵ in 100 μl) were pre-incubated for 15 minutes with varying concentrations of the chemoattractant at 37° with 5% CO2. Then 0.8uM of the recombinant serum amyloid A1 peptide (rSAA1, PeproTech, Cat#300-53) was added to the bottom chamber of each HTS transwell-96well plate (Coming#3387). Migration was induced by placing chemoattractant and the dHL60 cells mixture in the top chamber of the transwell plate. Cells were allowed to migrate for 120 min across the 5micron filters at 37° with 5% CO₂. Following migration, neutrophils or dHL-60 cells remaining in the transwell lower chamber (migrated fraction) were quantitated using the cell-titer-glo luminescence cell viability assay (Promega, G7571).

Enhancement of phagocytosis. Macrophages were elicited to the peritoneum of five C57BL6 mice by peritoneal injection of 1 ml of 1% Biogel in PBS (−/−) 4 days prior to harvest. Peritoneal exudates are harvested, combined and then filtered to remove Biogel beads. First, through a 70 um cell strainer followed by successively filtering through two 40 um cell strainers. The exudate is diluted with 1× PBS (−/−) to 50 ml and centrifuged at 300× g for 10 minutes at 4° C. The cell pellet is gently resuspended in 20-30 ml 1× PBS(+/+) and cells are counted using the Nexelcom Cellometer counter. Cell concentration is adjusted to 1,250,000 cells/ml in 1× PBS (+/+). 100 ul (125 k) cells are placed into each well of a 96-well Costar 3904 plate. The plates are centrifuged at 150× g for 30 seconds to promote adherence. After 90 minutes incubation at 37° C./5% CO₂, non-adherent cells are aspirated and attached macrophages (˜50K) are washed once with 150 ul 1× PBS (−/−) and then incubated overnight at 37° C./5% CO₂, in 135 ul pre-warmed serum-free Macrophage SFM/1× Pen-Strep media. The following day, 15 ul of freshly prepared 10× compound in serum-free Macrophage SFM media is added to each well, mixed and incubated for 15 minutes at 37° C./5% CO₂. Phagocytosis is initiated by the addition of a 10-fold excess (4 ul of 125 K/ul) of opsonized FITC Zymosan particles (Life Technologies). Phagocytosis is allowed to proceed for 45 minutes at 37° C./5% CO₂. Wells are aspirated, phagocytosis is arrested with 150 ul of ice-cold 1× PBS (−/−)/2 mM EDTA and aspirated again. Fluorescence signal from non-ingested Zymosan particles is quenched with 150 ul ice-cold 1:15 diluted Trypan Blue solution for 2 minutes and then aspirated to remove. Lastly, the plate is read on a SpectraMAX Gemini EM fluorescence plate reader in 150 ul of 1:50 diluted Trypan Blue. Plate Reader Settings=Bottom Read: Excitation 493 nm: Emission 525 nm: Cutoff 515 nm: Automix Off: Calibrate On: PMT=Auto: Column Priority: Reads/Well=20.

FPR2/ALX agonists for Heart Failure. Activation of the FPR2/ALX by endogenous small pro-resolution mediators (SPMs) such as Lipoxin A₄ (LXA₄), aspirin triggered 15-epi-LipoxinA₄ (ATL) and resolvin D1 (RvD1) as well as a synthetic small molecule ligands such as COMPOUND 1 results in stimulation of the non-phlogistic recruitment of monocytes and activation of macrophages in a manner that enhances the efferocytosis of apoptotic cells and promotes the clearance of necrotic cell debris. Stimulation of the FPR2/ALX activity also results in suppression of neutrophil recruitment. Activation of both mechanisms is proposed to be required for enhancement of wound healing mechanisms and returning of the injured heart to the homeostasis.

Preclinical in vitro Pharmacology of Compound 1. The FPR2/ALX natural pro-resolution ligands, such as lipoxinA₄, binds to the receptor triggering a wide array of cytoplasmatic cascades such as the Gi coupling, Ca²⁺ mobilization and P-arrestin recruitment. Activation of the FPR2/ALX by lipoxinA₄ modifies effects of peptidic agonists, such as serum amyloid A (SAA), and has alternative effects on phosphorylation pathways depending on cell type. In neutrophils, lipoxins modulate their movement, cytotoxicity and life span. In macrophages, lipoxins prevent their apoptosis and enhance efferocytosis. In most of inflammatory cells, lipoxins also down-regulate expression of several pro-inflammatory cytokines, such as IL-6, IL-1β and IL-8 as well as up-regulate expression of anti-inflammatory cytokine IL-10. Primary effects of lipoxin on neutrophils and macrophages are thought to be responsible for both termination of inflammation and initiation of resolution of inflammation. The latter is primarily responsible for the enhanced anti-fibrotic wound healing and returning of the injured tissue to the homeostasis. Compound 1 is a small molecule agonist of the FPR2/ALX which is thought to promote wound healing through enhancing the resolution of inflammation similarly to the FPR2/ALX natural SPMs.

Compound 1 was tested in following in vitro cell based assays. In the CHO-A12 cell lines over-expressing human FPR2/ALX (hFPR2/ALX) and human FPR1 (hFPR1) receptors, Compound 1 was a potent (50 nM) activator of the hFPR2/ALX Gi coupling resulting in lowering of the cAMP trough adenylcylase inhibition. Compound 1 was also an equally potent (10 nM) activator of the closely related hFPR1 receptor. In CHO-A12 cell lines over-expressing two mouse orthologs, mFPR2 and mFPR3, of the single hFPR2/ALX, Compound 1 was a very potent (20 nM) activator of mFPR2/ALX with no activity against mFPR3 (>10,000 nM). Similarly in human hFPR1, Compound 1 was non-selective with function affinity of approximately 50 nM with mFPR1 receptor. In neutrophil like human HL60 cell line, the Compound 1 potently (50 nM) increased the cytosolic Ca²⁺ levels. Compound 1 also stimulated recruitment of β-arrestin with potency of 3100 nM in DiscoveRx Pathhunter CHO-K1 hFPR2/ALX cell line.

Modulation of the cytosolic calcium mobilization in neutrophils and the cAMP levels in macrophages has been associated with either cellular movement (chemotaxis) or enhancement of phago-efferocytosis, respectively. Both of these activities are essential for compound classification as pro-resolution agonist of the FPR2/ALX receptor. Using human HL60 cell line Compound 1 stimulated chemotaxis, by itself, with potency of 78 nM. Compound 1 also antagonized chemotaxis induced by SAA with affinity of 189 nM. In mouse bio-gel elicited peritoneal macrophages, Compound 1 in picomolar range enhanced phagocytosis of the fluorescently labeled zymosan by between 250 to 60% pending on experimental conditions as compared to untreated control cells. This compound showed no such enhancement in bio-gel elicited peritoneal macrophages isolated from either single mFPR2 and mFPR3 or double mFPR2/FPR3 knockout mice.

Animal models. Permanent coronary artery occlusion was carried out in mice using a ligature placed around the left anterior descending artery to induce myocardial infarction. Treatment with orally-administered Compound 1(1 and 10 mg/kg; QD) or dosing solution without compound (QD, referred to as vehicle) was initiated 24 hours following myocardial infarction. Mice subjected to thoracotomy but not infarcted were included as surgical “sham” controls. Mice were evaluated 28 days following myocardial infarction to assess structure/function relationships. Hearts were removed from mice to evaluate the passive mechanics of the myocardium. To do this, ex vivo pressure-volume relationships of the left ventricle were measured via inflation and deflation cycles of a balloon placed within the left ventricle of the excised heart. Two-dimensional strains of the myocardial scar were also measured to determine the compliance of infarcted tissue. Hearts were also processed histologically to measure left ventricular dimensions, infarct areas and infarct collagen composition.

To assess myocardial fibrosis, mice were challenged with angiotensin II to stimulate cardiac hypertrophy and left ventricular collagen deposition. Mice were administered angiotensin II using subcutaneously implanted osmotic mini-pumps (˜2 mg/kg/day) A separate group of mice were implanted with subcutaneous pumps containing saline (surgical “sham” group); these mice served as control for pump implantation surgery. Depending on the specific study design, mice were treated with Compound 1 (1 and 10 mg kg; QD) or dosing solution without compound (QD, referred to as vehicle) either 24 hours before angiotensin II pump implantation, concurrent with pump implantation or 3 days following pump implantation. Treatments lasted for 2-3 weeks, depending on the exact study design. At the end of treatment phase, hearts were removed from animals and evaluated for collagen levels/fibrosis using a standard colorimetric assay for myocardial hydroxyproline or by cross-sectional histology of the hearts.

In both models, the following endpoints supporting the FPR2/ALX role in resolution of inflammation and enhancement in heart healing were observed.

Treatments were well tolerated throughout the in-life phase and no untoward effects on the physiology of the mice were noted. Mice treated at the high dose showed a decrease in overall mortality suggestion a survival benefit with treatment.

Treatment with Compound 1 preserved the normal compliance properties of myocardium as determined by measurements of ex vivo passive mechanics of the left ventricle. At the end of the treatment phase, hearts were arrested in diastole with a high potassium-containing cardioplegic solution. A modified balloon catheter assembly was placed into the left ventricle and balloons were inflated and deflated to measure pressure-volume relationships and the passive compliance properties of the left ventricular myocardium. Pressure-volume curves of mice treated with Compound 1 were left shifted in a dose-dependent manner indicating reduced left ventricular volumes. Smaller left ventricular volumes with Compound 1 treatments indicate less post infarction remodeling. The pressure-volume slopes of Compound 1 treated mice were greater than vehicle and similar to normal sham control mice indicating increased stiffness of the myocardium vs. vehicle and preservation of normal compliance properties similar to non-infarcted sham controls.

Two-dimensional scar strains (i.e., distensibility) were measured with a digital video camera concomitant with the pressure-volume measurements. Treatment with Compound 1 reduced circumferential and longitudinal strains relative to vehicle treatment indicating increased stiffness of the scar and less propensity for scar expansion. Strains were similar to normal sham control hearts indicating preservation of the normal compliance of the healed scar.

Histological evaluation of the hearts revealed reductions in left ventricular chamber area with Compound 1 treatment. Chamber areas were reduced to levels that approximated non-infarcted sham (28-30% reduced at 1 and 10 mg/kg, vs vehicle treated hearts, respectively; p<0.05).

Histological evaluation of left ventricular wall thickness at the site of infarction (anterior left ventricular free wall) revealed increased wall thicknesses with Compound 1 treatment relative to vehicle. Average anterior wall thickness values approached levels observed with non-infarcted shams indicating preservation of myocardial integrity (45-65% increased wall thickness vs vehicle, p<0.05).

Infarct area measured by histology (as a % of left ventricle area) was decreased with Compound 1 (44-49% reduced with 1 and 10 mg/kg vs. vehicle, respectively; p<0.05). The data suggest that treatment with Compound 1 reduces infarct expansion and infarct wall thinning following myocardial infarction.

Myocardial fibrosis was evaluated in the mouse with continuous angiotensin II challenge administered by subcutaneous osmotic mini-pump.

The effects of pre-treatment with Compound 1 on myocardial fibrosis was tested by treating mice orally by gavage 24 hours before angiotensin II challenge. This design is structured to evaluate prevention of fibrosis. Treated mice were dosed daily by oral gavage for 2 weeks. Treatment groups consisted of low dose and high dose Compound 1, vehicle control and an untreated sham group without angiotensin II challenge. Hearts were evaluated for collagen deposition following two weeks of concurrent treatment with Compound 1 and angiotensin II challenge. Left ventricular hydroxyproline content, measured as a surrogate of collagen, was decreased with Compound 1 treatment relative to control (83% with 1 mg/kg and 75% with 10 mg/kg vs. vehicle, p<0.05). Levels approached those measured in normal unchallenged hearts taken from the sham group. Comparable reductions in interstitial collagen were noted by histology of the left ventricle. The data indicate that FPR2/ALX agonists can attenuate myocardial fibrosis.

When treatment with Compound 1 was given at the time of angiotensin II challenge, comparable reductions in both hydroxyproline content and interstitial collagen levels by histology were observed.

Treatment with Compound 1 also reduces myocardial fibrosis when given after the development of myocardial fibrosis. This design is structured to evaluate the capacity of Compound 1 to ameliorate myocardial fibrosis as an interventional therapy. Mice challenged with angiotensin II for 3 days to develop fibrosis were treated with Compound 1 for 2.5 weeks in the setting of ongoing angiotensin II exposure. At the end of the treatment phase, hearts were evaluated by histology. Compound 1 treatment reduced interstitial fibrosis in the left ventricle relative to vehicle (˜74% reduction vs. vehicle p<0.001). Fibrosis levels were comparable to those measured in the untreated sham group without angiotensin II challenge.

It will be evident to one skilled in the art that the present disclosure is not limited to the foregoing illustrative examples, and that it can be embodied in other specific forms without departing form the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

We claim:

1. A method for treating heart disease comprising administering a therapeutically effective amount of an FPR2/ALX agonist to a patient in need thereof

2. The method of claim 1 wherein the heart disease is selected from the group consisting of angina pectoris, unstable angina, myocardial infarction, heart failure, acute coronary disease, acute heart failure, chronic heart failure, and cardiac iatrogenic damage.

3. The method of claim 1 wherein the treatment is post myocardial infarction.

4. The method of claim 1 wherein the treatment is associated with chronic heart failure.

5. The method of claim 1 where the treatment is to improve myocardial wound healing.

6. The method of claim 1 where the treatment is to diminish myocardial fibrosis.

7. The method of claim 1 where the agonist is 1-(4-chlorophenyl)-3-(5-isopropyl-1-methyl-3-oxo-2-phenyl-2,3-dihydro-1H-pyrazol-4-yl)urea or a pharmaceutically acceptable salt thereof.
